(19) United States
(12) Patent Application Publication
(10) Pub. No.: US 2018/0053297 A1
(43) Pub. Date: Feb. 22, 2018

(54) Methods and Apparatuses for Detection of Abnormalities in Low-Contrast Images

(71) Applicant: Ohio University, Athens, OH (US)

(72) Inventors: Mehmet Celenk, Athens, OH (US); Akshay S. Bharadwaj, Athens, OH (US)

(73) Assignee: Ohio University, Athens, OH (US)

(21) Appl. No.: 15/674,750

(22) Filed: Aug. 11, 2017

Related U.S. Application Data
(60) Provisional application No. 62/376,736, filed on Aug. 18, 2016.

(51) Int. Cl. G06T 7/00; G06T 7/11; G06K 9/62; G06K 9/46; A61B 6/00

(52) U.S. Cl. CPC G06T 7/0012; A61B 8/5223; G06K 9/6267; G06K 9/4604; G06K 9/6202; A61B 6/5217; A61B 6/502; G06T 2207/10116; G06T 2207/10088; G06T 2207/10132; G06T 2207/30024; G06T 2207/30096; G06T 2207/30068; G06T 2207/30101; G06T 7/11

(57) ABSTRACT

Devices and methods for determining abnormalities in cells or tissues in a subject from a captured image from the subject are described.

### CROSS-REFERENCE TO RELATED APPLICATIONS

This application claims the priority to U.S. Provisional Application Ser. No. 62/376,736, filed Aug. 18, 2016, the entire disclosure of which is expressly incorporated herein by reference.

### TECHNICAL FIELD

Described herein are methods and apparatuses for the detection of abnormalities in images that may be considered to be “low-contrast” images. In one particular aspect, it relates to a computer-assisted device and method for detection of abnormalities in cells, tissues and/or lesions in scanned images.

### STATEMENT REGARDING FEDERALLY FUNDED SPONSORED RESEARCH

This invention not was made with any government support and the U.S. government has no rights in the invention.

### BACKGROUND OF THE INVENTION

As one example where it is desired to improve information gained from scanned images, the diagnosis of breast cancer is of importance.

Breast cancer is the most common type of cancer among women with a high mortality rate. According to the research by American Cancer Society (ACS), there has been an estimate of 232,670 new cases in the United States of America alone in the year 2014, out of which the death count is more than 40,000. There are several causes of breast cancer and the most effective way to improve the success rate of treatment is its early detection. The vastly used diagnosis modality of breast cancer is through mammography, which has a high success rate in diagnosing radiography based cancer detection. The micro-calcifications (MCs) are detected on the mammogram as highly small bright spots, due to its high attenuation compare with the surrounding breast tissue. The MCs are the initial indicators of malignant lesion in the breast tissue, which can be diagnosed by the pattern in which they occur. The diameter of an MC can be as small as 0.1 mm occurring among the soft tissue. The region of occurrence has very high local luminance mean due to the direct reflection of the X-rays and decreased local contrast resulting from the attenuated rays falling on the X-ray film (see FIG. 1).

This makes it challenging to detect the MC. Although significant research effort has been devoted to detection and computer aided diagnosis (CAD) of the breast cancer, there still have been numerous complex problems that need to be addressed. Among them, the detected MCs should be classified into malignant or benign classes based on the distribution characteristics of the MCs.

In spite of considerable research into new methods to diagnose and treat this disease, breast cancer remains difficult to diagnose effectively, and the mortality observed in patients indicates that improvements are needed in the diagnosis, treatment and prevention of this disease.

### SUMMARY OF THE INVENTION

In a first broad aspect, there is described herein a detection system to detect and classify the abnormalities in radiographic images of cell, tissues and/or lesions.

Described herein is a method for determining whether a subject has an abnormality present in a cell or tissue, which includes the steps of:
i) determining at least one region-of-interest in an image taken of the subject using an image segmentation procedure; and
ii) classifying the image of step i) using a clique pattern procedure to determine the presence or absence of patterns among at least a first pixel and its adjacent pixels in the region-of-interest in the segmented image;
and, based on the presence or absence of such clique patterns, determining whether the subject has such abnormality

Also described is a computer-implemented method for determining whether a subject has an abnormality present in a cell or tissue, where at least a portion of the method is performed by a computing device comprising at least one processor.

Also described is a method where the image segmentation procedure comprises using a progressive segmentation of the image to separate the region-of-interest from background in the image.

Also described is a method where the progressive segmentation comprises using: Fuzzy C-Means Clustering (FCM) which allows data to have different degrees of membership with each clusters; and, White Top-Hat transform which creates different intensity profiles in the image and allows for performing histogram based thresholding.

Also described is a method where the clique pattern procedure comprises using a Gibbs Random Fields (GFRs) clique pattern extraction to search for patterns in the region-of-interest in the image.

Also described is a method where the image includes one or more of: radiographic (X-Ray) images, computer axial tomography (CAT) scans, magnetic resonance images (MRI), and ultrasonic images.

Also described is a method where the abnormality is one or more of: micro-calcifications (MCs), tumors, lesions, injury, tear, or other damage to the tissue or organ.

Also described is a method where the tissues include one or more of blood vessels such as small and large arteries, heart valves; joints and tendons, such as knee joints and rotator cuff tendons; soft tissues such as breast, thyroid, testes, muscle, and fat; organs such as brain, kidney, bladder, and gallbladder.

Also described is a method that further includes indicating when a therapeutic intervention aimed is beneficial.

Also described is a method further including a step of correlating the data with similar data from a reference population.

Various objects and advantages of this invention are apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1. Images showing a normal mammogram (left) and a mammogram with calcifications (right).

FIG. 2. Schematic illustration of a system for modification of local contrast as a function of luminance mean.

FIGS. 3A-3D. Diagrams showing: (FIG. 3A) two pixel cliques; (FIG. 3B) and (FIG. 3C) three pixel cliques; and, (FIG. 3D) four pixel clique.

FIG. 4. Diagram showing a 7×6 image window from a test image which contains a micro-calcification (MC).

FIGS. 5A and 5B. Images showing: FIG. 5A—original mammogram with calcification; and, FIG. 5B -segmented mammogram with isolated breast region.

FIGS. 6A and 6B. Images showing: FIG. 6A—top-hat transform of the segmented image; and, FIG. 6B—histogram of the top-hat transformed image.

FIGS. 7A and 7B. Images showing: FIG. 7A—resultant image of top-hat transform after histogram thresholding; and FIG. 7B—watershed segmentation of the top-hat transformed image.

FIG. 8. Image showing ROI extracted after watershed segmentation.

FIGS. 9A and 9B. Images showing: FIG. 9A - result of searching for multi-pixel cliques; and FIG. 9B—result of detection of MCs after the search for multi-pixel cliques and single pixel cliques.

FIG. 10A. Table I summarizes the findings for 6 images with MCs used for testing the method.

FIG. 10B. Block diagram illustrating one example of a hardware implementation for the methods described herein.

FIGS. 11A-11G. (11A) Original Image; (11B) Top-hat Transform of the segmented image; (11C) Histogram of the top-hat transformed image; (11D) Shows resultant image of top-hat transform after histogram thresholding is performed; (11E) Watershed segmentation of the top-hat transformed image, (11F) ROI extracted; and, (11G) Micro-calcifications detected.

FIGS. 12A-12G. (12A) Original Image; (12B) Top-hat Transform of the segmented image; (12C) Histogram of the top-hat transformed image; (12D) Shows resultant image of top-hat transform after histogram thresholding is performed; (12E) Watershed segmentation of the top-hat transformed image, (12F) ROI extracted; and, (12G) Micro-calcifications detected.

FIGS. 13A-13G. (13A) Original Image; (13B) Top-hat Transform of the segmented image; (13C) Histogram of the top-hat transformed image; (13D) Shows resultant image of top-hat transform after histogram thresholding is performed; (13E) Watershed segmentation of the top-hat transformed image, (13F) ROI extracted; and, (13G) Micro-calcifications detected

FIGS. 14A-14G. (14A) Original Image; (14B) Top-hat Transform of the segmented image; (14C) Histogram of the top-hat transformed image; (14D) Shows resultant image of top-hat transform after histogram thresholding is performed; (14E) Watershed segmentation of the top-hat transformed image; (14F) ROI extracted; and, (14G) Micro-calcifications detected

FIGS. 15A-15G. (15A) Original Image; (15B) Top-hat Transform of the segmented image; (15C) Histogram of the top-hat transformed image; (15D) Shows resultant image of top-hat transform after histogram thresholding is performed; (15E) Watershed segmentation of the top-hat transformed image; (15F) ROI extracted; and, (15G) Micro-calcifications detected.

### DETAILED DESCRIPTION OF THE PREFERRED EMBODIMENT(S)

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Definitions

Images: A generic term that includes one or more of the following: radiographic (X-Ray) images, computer axial tomography (CAT) scans, magnetic resonance imaging (MRI), and ultrasonic images.

Therapeutic: A generic term that includes both diagnosis and treatment. It will be appreciated that in these methods the “therapy” may be any therapy for treating a disease including, but not limited to, pharmaceutical compositions, gene therapy and biologic therapy such as the administering of antibodies and chemokines. Thus, the methods described herein may be used to evaluate a patient before, during and after therapy, for example, to evaluate the reduction in disease state.

Adjunctive therapy: A treatment used in combination with a primary treatment to improve the effects of the primary treatment.

Clinical outcome: Refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Decrease in survival: As used herein, “decrease in survival” refers to a decrease in the length of time before death of a patient, or an increase in the risk of death for the patient.

Patient: As used herein, the term “patient” includes human and non-human animals. The preferred patient for treatment is a human. “Patient,” “individual” and “subject” are used interchangeably herein.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Poor prognosis: Generally refers to a decrease in survival, or in other words, an increase in risk of death or a decrease in the time until death. Poor prognosis can also refer to an increase in severity of the disease, such as an increase in spread (metastasis) of the cancer to other tissues and/or organs.

Screening: As used herein, “screening” refers to the process used to evaluate and identify candidate agents that affect such disease.

General Description

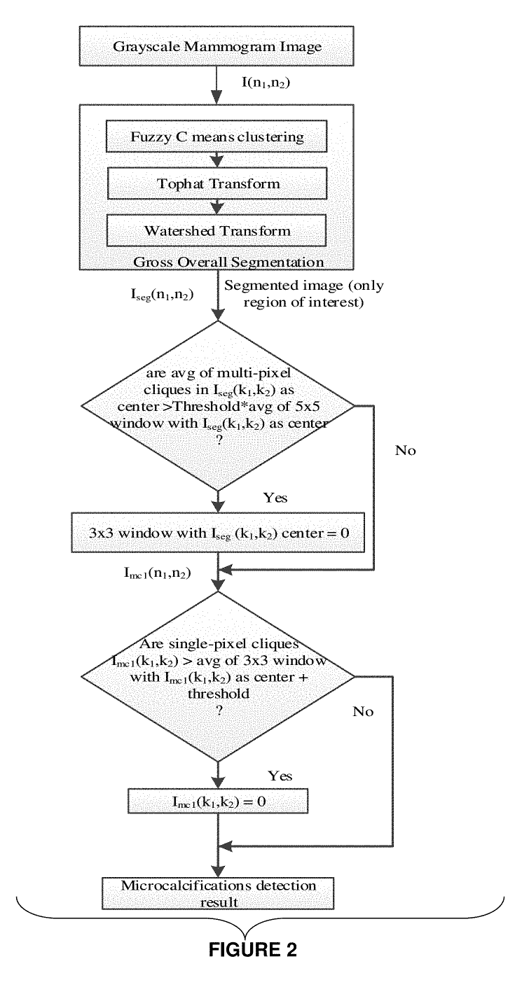

Thus, the present method provides a system for the modification of local contrast as a function of luminance mean. One example of such method is generally shown in the block diagram as shown in FIG. 2.

While the presently described methods and apparatuses are useful the detection of different types of abnormalities (e.g., micro-calcifications (MCs), tumors, lesions, injury, tear, or other damage to the tissue or organ), in images taken by various detection means (e.g., radiographic (X-Ray) images, computer axial tomography (CAT) scans, magnetic resonance imaging (MRI), and ultrasonic images), the following description is one exemplary embodiment of the present invention.

### Exemplary Embodiment

Image Segmentation

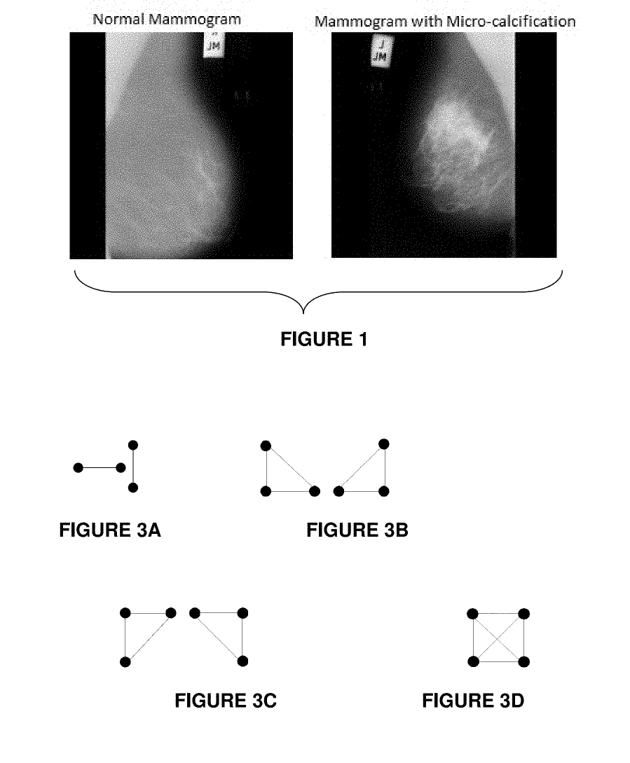

Referring again to FIG. 1, in the mammograms, it can be seen that there are two distinct regions, a region-of-interest (ROI) where there may be an abnormality (i.e., the breast region) and the background dark area which is of no interest. Considering only the ROI or the breast region with high probability of finding MCs enables the method described herein to reduce the computational cost involved in processing high resolution X-ray mammogram. That is, the image is first segmented into the breast region and the remaining part of the image. The segmentation of a mammogram is challenging due to the reduced contrast between the background and the breast skin. This is carried out by the Fuzzy C Means (FCM) clustering to isolate the breast region.

FCM clustering is a robust segmentation algorithm that allows the data to belong to multiple clusters with different degrees of memberships with each cluster. The algorithm solely works on minimization of the clustering error function Jₘ

$${J}_{m} = {\sum }_{i = l}^{N} {\sum }_{j = l}^{C} {u}_{ij}^{m} {‖ {x}_{i} - {c}_{j} ‖}^{2} \qquad ( 1 )$$

where the range of real number m is 1≦m<∞, the uᵢⱼ is the degree of membership of xᵢ in the j cluster, xᵢ is the i^{th} of the d-dimensional measured data, cⱼ is the d-dimensional center of the cluster, and ∥*∥ is the Euclidian norm expressing the similarity between any measured data and the center.

The minimization of the above function is an iterative procedure where the optimization occurs while updating the memberships uᵢⱼ and the cluster centers cᵢⱼ using the relation in Equations (2) and (3). The minimization of Jₘ is achieved only when the value of uᵢⱼ stops changing significantly and has reached its steady state limit. The saturation criterion can be given by Equation (4).

$$\begin{matrix}Uij = \frac{1}{{\sum }_{k = l}^{c} {( \frac{‖ {x}_{i} - {c}_{j} ‖}{‖ {x}_{i} - {c}_{k} ‖} )}^{\frac{2}{m - l}}} , and & ( 2 ) \\ {c}_{j} = \frac{{\sum }_{j = l}^{N} {u}_{ij}^{m} \cdot {x}_{i}}{{\sum }_{j = l}^{m} {u}_{ij}^{m}} & ( 3 ) \\ {max}_{ij} \{ \vert {u}_{ij}^{( k + 1 )} - {u}_{ij}^{( k )} \vert \} < ɛ & ( 4 )\end{matrix}$$

where ε is a number between 0 and 1 and k is the iteration index.

FCM considers all the pixels of the mammograms as a dataset. All the data points are divided c clusters. An appropriate level of cluster fuzziness ‘m’ is considered as a real value greater than 1. A membership matrix U=(uⱼₖ)_{c×n} is initialized where uᵢⱼε[0, 1] and

$${\sum }_{j = 1}^{c} {u}_{ij} = 1 ; \\ j = 1 , 2 , 3 , … n \qquad ( 5 )$$

The centers of the cluster cᵢⱼ are calculated using Equation (3). For the sake of simplicity, m=2 and c=5. This allows the C-means algorithm to classify the pixels into five different regions or clusters based on the intensity profile of the image. The first region is the unwanted background area which is dark. Progressively the pixels are classified from one to five with label five for the pixels with the higher intensities. The clusters labeled four and five are considered for further analysis, as they contain the information necessary for further processing.

The FCM segmented image still contains unwanted regions such as the region surrounding the dense soft tissue, part of the thoracic region appearing on the mammogram and the x-ray label which will interfere with the image analysis operations. To eliminate of the unwanted areas, the image is further segmented using white top-hat transform. The white top-hat transform of the image is defined as difference of the image and its openings, as given in Equation 6. TH [I (n₁, n₂)] is the top-hat transform of the image, I(n₁,n₂) is the input image and γ(I(n₁, n₂)) is the opening of the image.

TH[I(n₁, n₂)]=I(n₁, n₂)−γ(I(n₁, n₂)) (6)

The opening of the image is defined as the erosion of set A by set B, where set A and B belong to a 2-D integer space X², followed by the dilation of the previously calculated result by B. It is represented as

A_{∘}B=(A⊖B)⊕B (7)

Dilation of A by B is the set of all displacements x such that B and A overlap by at least 1 element, where B is the reflection of B by its origin and shifted by x.

A⊕B={x|[(B)ₓ∩A]⊂A} (8)

Erosion of set A by B can be defined as all the points x such that the B translated by x is present in A. The erosion equation is given by

A⊕B={x|(B)ₓCA} (9)

The structuring element used is a disk structure with a radius of 18 pixels. The top-hat transform detects the areas with lower intensity and enhances the areas; whereas, areas with high intensity and low contrast are attenuated. This difference is shown in a histogram, which allows to perform a histogram based threshold between these two areas to result in an image with reduced region of interest and other non-related areas. The isolation of the ROI can be done in several ways. In this embodiment, a marker controlled watershed segmentation is used to capture only the ROI (see FIG. 5B). The ROI is further processed to detect the MCs.

Pattern Recognition using the Gibbs Random Fields (GRFs) and Thresholding

Since the MCs do not have a specific shape and occurrence pattern, the MCS can appear in random shapes and orientations. To detect the patterns created by the MCs, the relation of the Markov Random Fields (MRFs) and the Gibbs distribution, called the GRFs, are used. The Gibbs conditional probability is given by

$$P ( {ω}_{ij} \vert {N}_{ij} ) = \frac{1}{Z} exp ( - \frac{1}{T} {\sum }_{k}^{ } {F}_{k} ( {C}_{k} ( i , j ) ) ) \qquad ( 10 )$$

where Z is the normalizing function, T is the parameter, Fₖ( ) is the function of states of the pixels in the cliques, Cₖ(i,j) are cliques in the image, and Nᵢⱼ is the defined neighborhood around ωᵢⱼ. Cliques are the patterns among a pixel and its neighbor that can be observed in a given neighborhood. A clique can contain a single pixel or multiple pixels. The search for finding the cliques can be carried out using four connectivity or eight connectivity. In this example, eight connectivity is employed. All the possible cliques that can appear in an image are given in FIGS. 3A-3D where there are shown: (FIG. 3A) Two pixel cliques, (FIG. 3B) and (FIG. 3C) three pixel cliques, (FIG. 3D) four pixel clique.

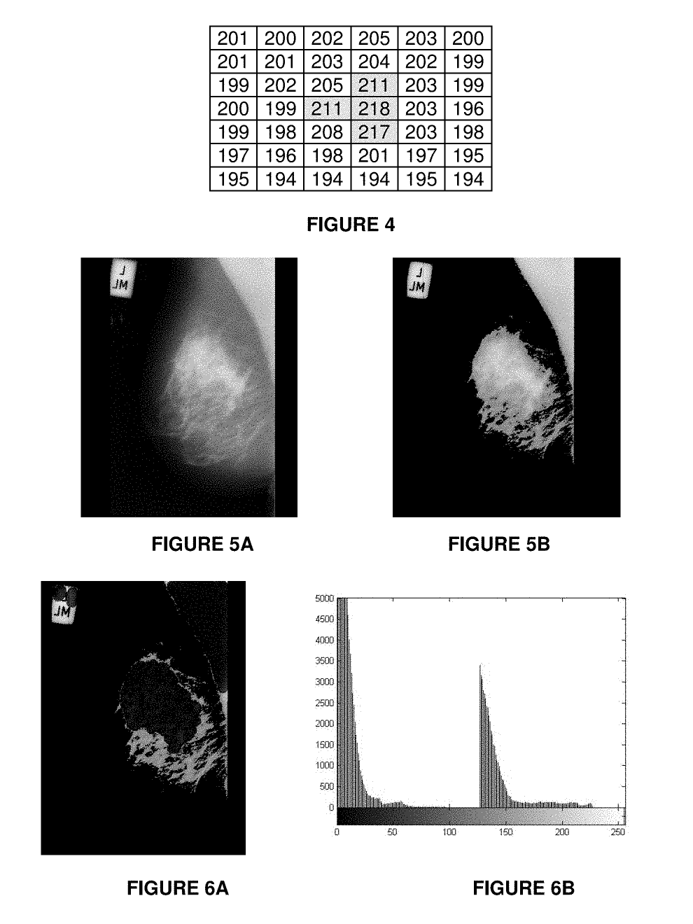

To illustrate the concept, consider a 7×6 local window in FIG. 4, selected from the test image. From FIG. 4, it can be clearly seen that the pixels in the center has a significantly higher intensity values compared to the background and hence represents an MC pattern.

Observing the texture pattern that the center point forms with the neighborhood, the pattern can represented by two ‘L’ shaped cliques with different orientations from FIG. 3C. Similarly, all the MCs in the image can be characterized as a combination of one or more such cliques as given in FIGS. 3A-3D.

In order to detect the MCs, the average intensity of the pixels in a clique (3×3 pixel window) is calculated as In order to detect the MCs, the average intensity of the pixels in a clique is calculated in accordance with FIG. 3B and FIG. 3C as

$$\begin{matrix}\hat{I} ( {n}_{1} , {n}_{2} ) = \frac{1}{3} [ I ( {n}_{1} , {n}_{2} ) + I ( {n}_{1} , {n}_{2} + 1 ) + I ( {n}_{1} - 1 , {n}_{2} + 1 ) ] & ( 11 ) \\ \hat{I} ( {n}_{1} , {n}_{2} ) = \frac{1}{3} [ I ( {n}_{1} , {n}_{2} ) + I ( {n}_{1} , {n}_{2} + 1 ) + I ( {n}_{1} + 1 , {n}_{2} + 1 ) ] & ( 12 ) \\ \hat{I} ( {n}_{1} , {n}_{2} ) = \frac{1}{3} [ I ( {n}_{1} , {n}_{2} ) + I ( {n}_{1} , {n}_{2} + 1 ) + I ( {n}_{1} + 1 , {n}_{2} ) ] & ( 13 ) \\ \hat{I} ( {n}_{1} , {n}_{2} ) = \frac{1}{3} [ I ( {n}_{1} , {n}_{2} ) + I ( {n}_{1} , {n}_{2} + 1 ) + I ( {n}_{1} - 1 , {n}_{2} ) ] & ( 14 )\end{matrix}$$

The resultant average clique intensity Î(m, n₂) is then compared to the weighted average intensity of the pixels in the 5×5 window enclosing the clique pattern in question. The weighted average intensity is calculated using Equation 15

$${I}_{T} = Threshold * \frac{1}{{5}^{2}} {\sum }_{{n}_{1} = - 2}^{2} {\sum }_{{n}_{1} = - 2}^{2} I ( {n}_{1} , {n}_{2} ) \qquad ( 15 )$$

The average intensity of the cliques is greater than a threshold times the average of the neighborhood. When this condition is satisfied, a threshold is performed on the cliques to indicate the detected MCs. A hard limiter is used to perform the detection.

$${MC}_{i} = \{ \begin{matrix}1 , & if \hat{I} ( {n}_{1} , {n}_{2} ) > {I}_{T} \\ 0 , & otherwise\end{matrix} \qquad ( 16 )$$

where i=1, 2, 3, 4 representing 4 different clique patterns as in FIG. 3B and FIG. 3C. Furthermore, the small MCs which are in the high intensity areas are missed when searching for MCs using multi-pixel cliques. Thus single pixel cliques are adopted and by examination of such regions, it is found that the intensity of the pixel in the clique is greater than the sum of the average of pixels in the neighborhood of 3×3 window and threshold value.

Thus, a thresholding is performed in the selected areas shown in FIG. 4, where the above condition is satisfied.

### EXAMPLES

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

For this Example, a subset of the database with 322 mammograms from the digital mammogram database of the Mammographic Image Analysis Society was used; out of which 25 mammograms have calcifications and MCs. The remaining is a mixture of normal mammograms and mammograms with various disorders. In this example, fifty one mammograms were selected, with 25 images were with MC and the rest were normal.

As the first step, the image segmentation is achieved by applying the FCM to the original image with the degree of fuzziness set to two in order to obtain a crisp clustered image and the number of clusters is set to five. The unwanted background and the required region of interest are classified separately. White top-hat transform is performed on the segmented image (FIG. 5B).

The resulting top-hat transformed image is depicted in FIG. 6A, the histogram of the top-hat transformed image is shown in FIG. 6B.

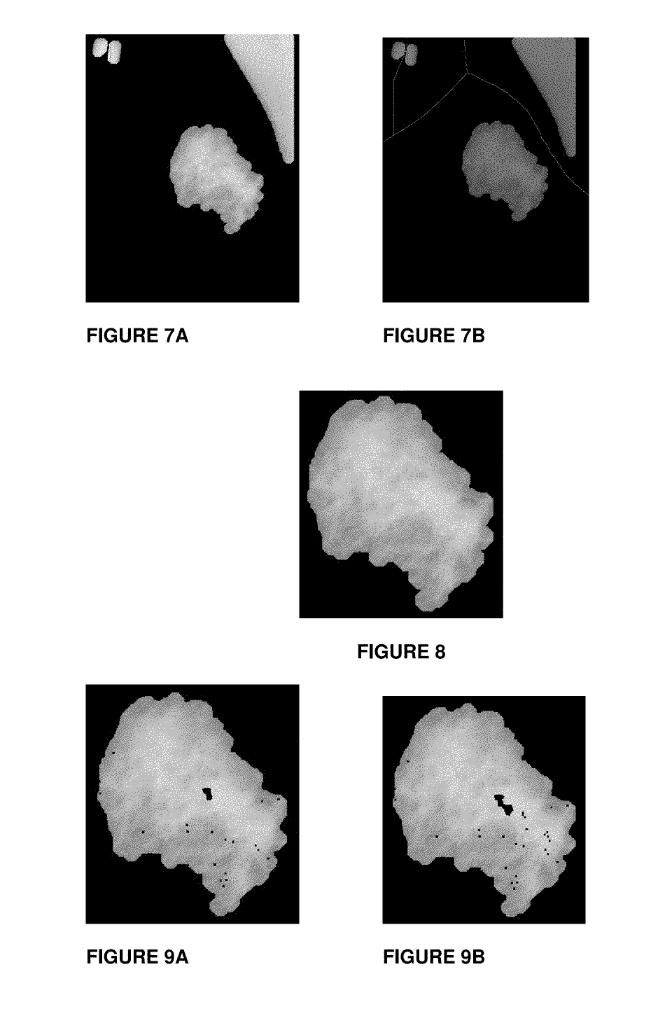

This segmentation isolates the ROI from the rest of the image as shown in FIG. 7A. Watershed segmentation is performed to separate the ROI from the rest (FIG. 7B).

Further processing is conveyed on the extracted ROI (see FIG. 8 for the extracted ROI). This saves computational time and hence makes the system computationally less demanding.

Referring now to FIGS. 9A-9B, FIG. 9A shows the result of searching for multi-pixel cliques, while FIG. 9B shows the result of detection of MCs after the search for multi-pixel cliques and single pixel cliques.

Thus, in the search of MCs, thresholding is performed on the 3×3 windows containing the cliques. The result of finding multi-pixel cliques in the ROI is given in FIG. 9A. The threshold used to detect the MCs in a multi-pixel clique is 1.047, which is empirically determined in this case. Since the patterns searched are for multi-pixel cliques, several small sized MCs are missed. Hence, a search for single pixel cliques is conducted. For this purpose a 3×3 window is considered, with the center pixel of the window as clique, if the intensity of the clique is greater than the sum of average of the 3×3 window and a determined threshold. The threshold is empirically determined to be 1.054. The results of searching for 1 pixel cliques are shown in FIG. 9B. The results of the search demonstrate high detection rate and low false negative rate. One of the drawbacks of the algorithm is that the algorithm misclassifies the overlapping of the soft tissues which appear bright, similar to MC but has a smooth appearance. Hence, this method has a slightly higher rate of false positive than desired.

The segmentation works efficiently in isolating the ROI in all the tested images. The overall detection rate (DR) is given by Equation (11), where FP is the false positive, TP is the true positive, FN is the false negative, and TN is the true negative. The detection rate of the proposed method is found to be 94.4%.

$$DR = \frac{FP + TP}{FP + TP + FN + TN} \qquad ( 17 )$$

The sensitivity (S) of the algorithm is measured by

$$S = \frac{TP}{TP + FN} \qquad ( 18 )$$

which is found to be 93.7% in this case. The rate of detection of false negatives in the image is 5.6%. The detection accuracy (DA) is given by

$$DA = \frac{TP}{TP + FP} \qquad ( 19 )$$

and it is determined to be 88.2%, respectively.

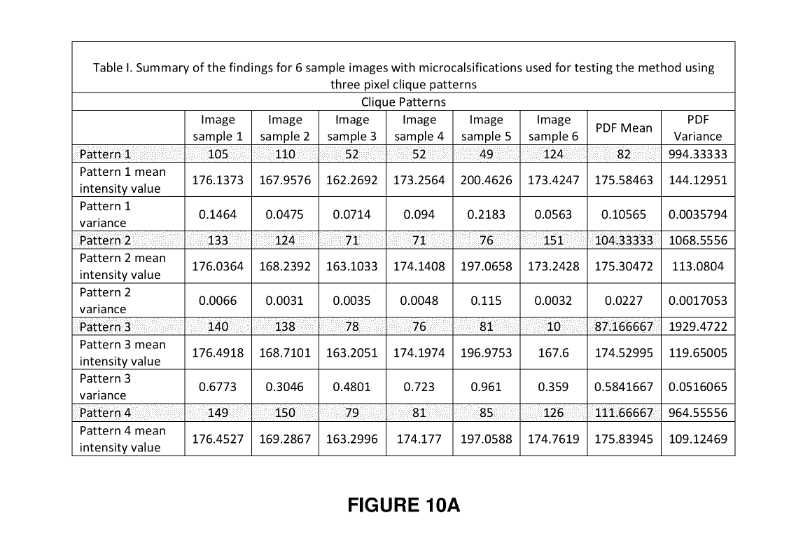

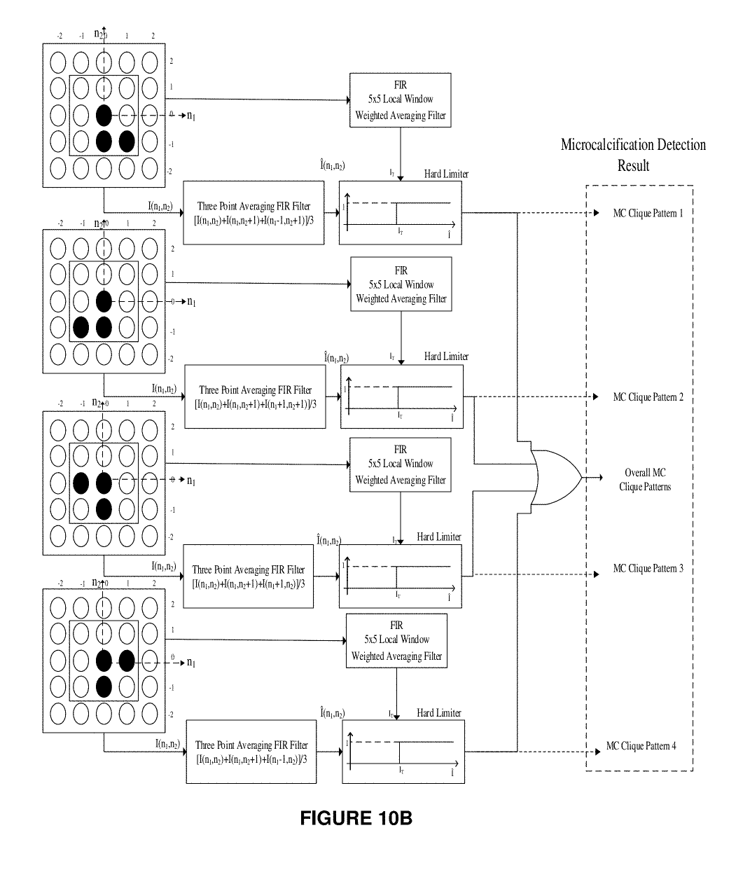

Table I in FIG. 10A summarizes the findings for 6 images with MCs used for testing the method. The findings in Table I—FIG. 10A are confined to three pixel cliques.

FIG. 10B shows a block diagram for the hardware implementation of one embodiment of the method described herein.

### Example 2

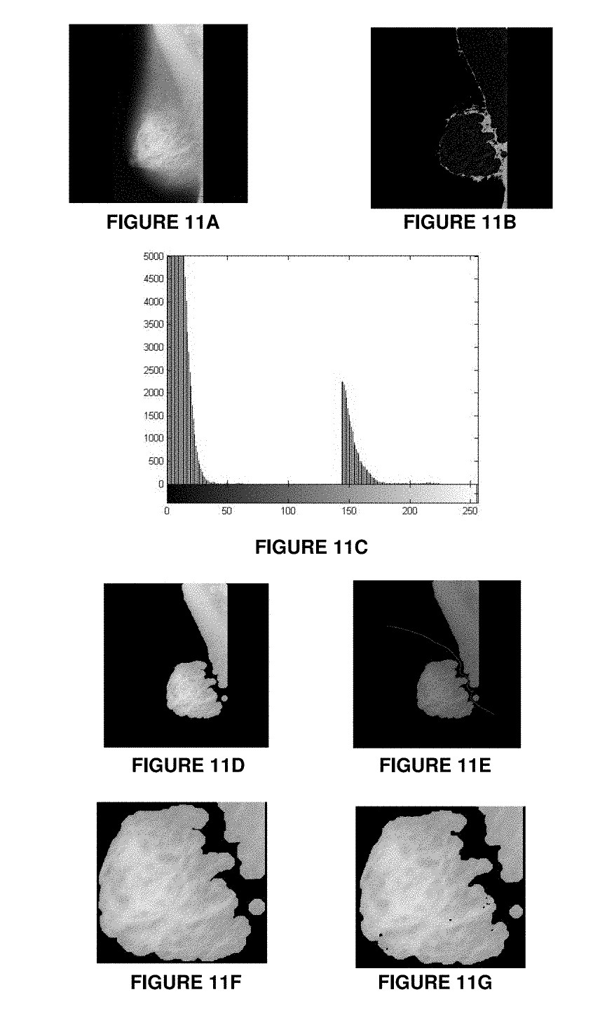

FIGS. 11A-11B. (11A) Original Image; (11B) Top-hat Transform of the segmented image; (11C) Histogram of the top-hat transformed image; (11D) Shows resultant image of top-hat transform after histogram thresholding is performed; (11E) Watershed segmentation of the top-hat transformed image, (11F) ROI extracted; and, (11G) Micro-calcifications detected.

### Example 3

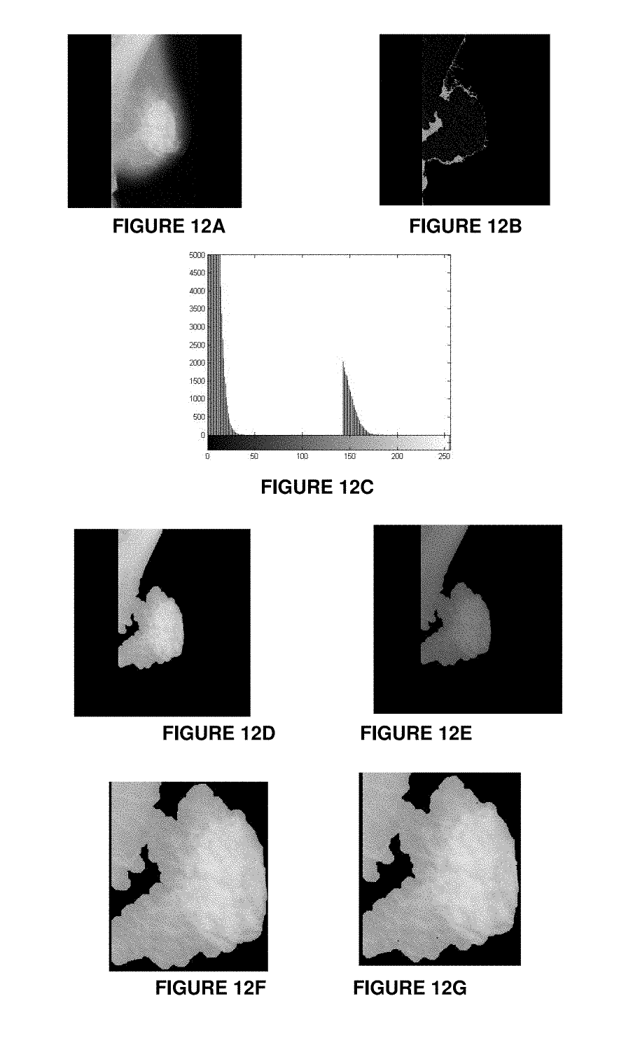

FIGS. 12A-12G. (12A) Original Image; (12B) Top-hat Transform of the segmented image; (12C) Histogram of the top-hat transformed image; (12D) Shows resultant image of top-hat transform after histogram thresholding is performed; (12E) Watershed segmentation of the top-hat transformed image, (12F) ROI extracted; and, (12G) Micro-calcifications detected.

### Example 4

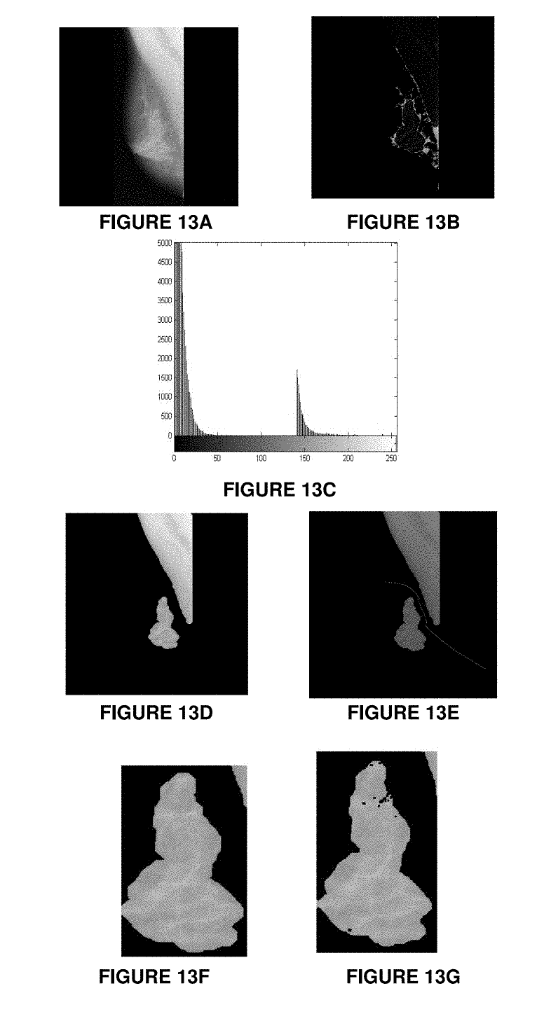

FIGS. 13A-13G. (13A) Original Image; (13B) Top-hat Transform of the segmented image; (13C) Histogram of the top-hat transformed image; (13D) Shows resultant image of top-hat transform after histogram thresholding is performed; (13E) Watershed segmentation of the top-hat transformed image, (13F) ROI extracted; and, (1G) Micro-calcifications detected.

### Example 5

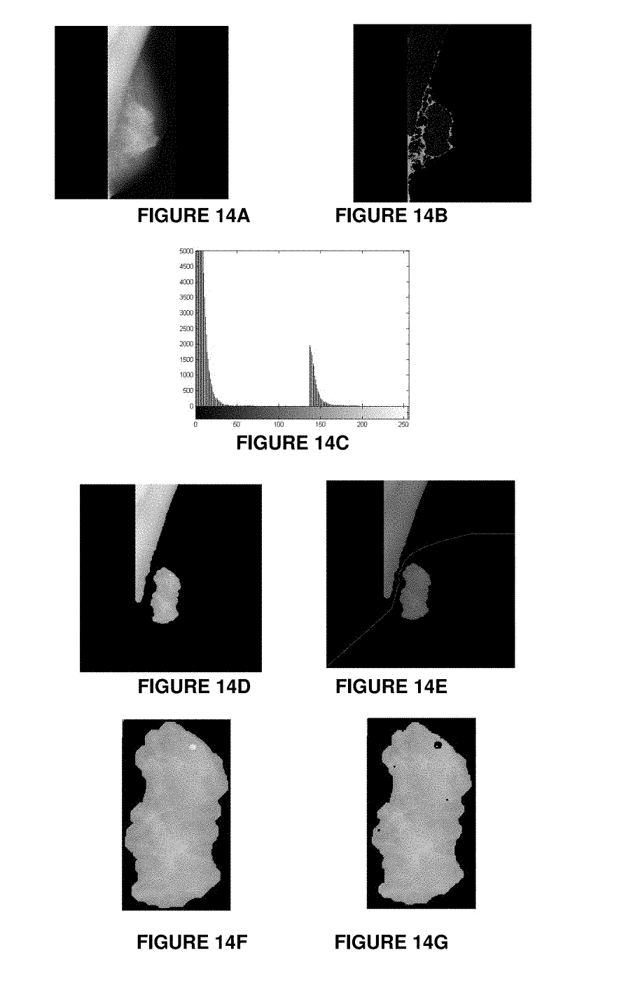

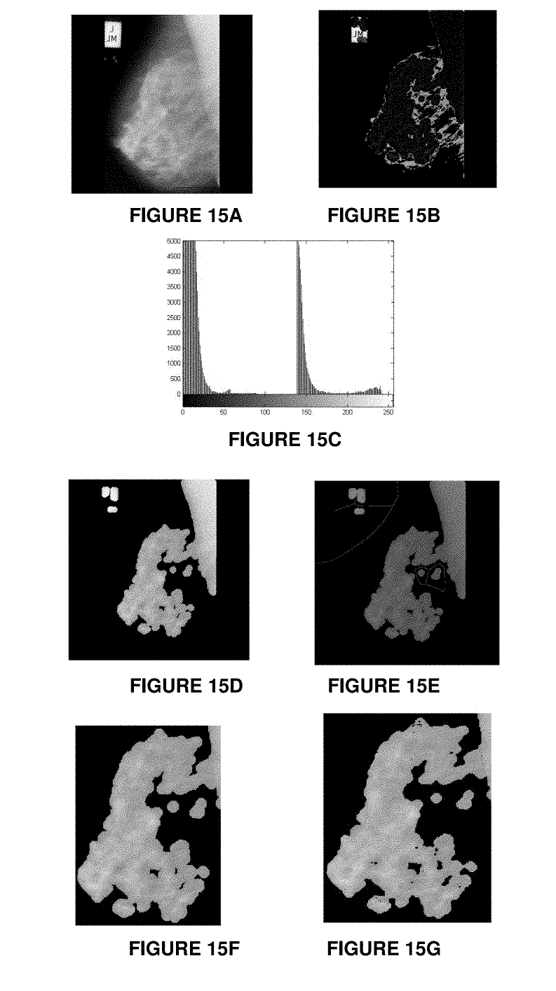

FIGS. 14A-15G. (14A) Original Image; (14B) Top-hat Transform of the segmented image; (14C) Histogram of the top-hat transformed image; (14D) Shows resultant image of top-hat transform after histogram thresholding is performed; (14E) Watershed segmentation of the top-hat transformed image; (14F) ROI extracted; and, (15G) Micro-calcifications detected.

### Example 6

FIGS. 15A-15G. (15A) Original Image; (15B) Top-hat Transform of the segmented image; (15C) Histogram of the top-hat transformed image; (15D) Shows resultant image of top-hat transform after histogram thresholding is performed; (15E) Watershed segmentation of the top-hat transformed image; (15F) ROI extracted; and, (15G) Micro-calcifications detected.

Non-limiting Examples of Electronic Apparatus Readable Media, Systems, Arrays and Methods of Using the Same

A “computer readable medium” is an information storage media that can be accessed by a computer using an available or custom interface. Examples include memory (e.g., ROM or RAM, flash memory, etc.), optical storage media (e.g., CD-ROM), magnetic storage media (computer hard drives, floppy disks, etc.), punch cards, and many others that are commercially available. Information can be transmitted between a system of interest and the computer, or to or from the computer to or from the computer readable medium for storage or access of stored information. This transmission can be an electrical transmission, or can be made by other available methods, such as an IR link, a wireless connection, or the like.

“System instructions” are instruction sets that can be partially or fully executed by the system. Typically, the instruction sets are present as system software.

The system can also include detection apparatus that is used to detect the desired information, using any of the approaches noted herein. For example, a detector configured to obtain and store images or a reader can be incorporated into the system. Optionally, an operable linkage between the detector and a computer that comprises the system instructions noted above is provided, allowing for automatic input of specific information to the computer, which can, e.g., store the database information and/or execute the system instructions to compare the detected specific information to the look up table.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Sequel™, Oracle™, Paradox™) can be adapted to the present invention. For example, the systems can include software having the appropriate character string information, e.g., used in conjunction with a user interface (e.g., a GUI in a standard operating system such as a Windows, Macintosh or LINUX system) to manipulate strings of characters. Specialized sequence alignment programs such as BLAST can also be incorporated into the systems of the invention.

As noted, systems can include a computer with an appropriate database. Software, as well as data sets entered into the software system comprising any of the images herein can be a feature of the invention. The computer can be, e.g., a PC (Intel x86 or Pentium chip-compatible DOS™, OS2™, WINDOWS™, WINDOWS NT™, WINDOWS95™, WINDOWS98™, WINDOWS2000, WINDOWSME, or LINUX based machine, a MACINTOSH™, Power PC, or a UNIX based (e.g., SUN™ work station or LINUX based machine) or other commercially common computer which is known to one of skill Software for entering and aligning or otherwise manipulating images is available, e.g., BLASTP and BLASTN, or can easily be constructed by one of skill using a standard programming language such as Visualbasic®, Fortran, Basic, Java, or the like.

In certain embodiments, the computer readable medium includes at least a second reference profile that represents a level of at least one additional image from one or more samples from one or more individuals exhibiting indicia of abnormalities.

In another aspect, there is provided herein a computer system for determining whether a subject has, or is predisposed to having, abnormalities, comprising a database and a server comprising a computer-executable code for causing the computer to receive a profile of a subject, identify from the database a matching reference profile that is diagnostically relevant to the individual profile, and generate an indication of whether the individual has abnormalities.

In another aspect, there is provided herein a computer-assisted method for evaluating the presence, absence, nature or extent of abnormalities degeneration in a subject, comprising: i) providing a computer comprising a model or algorithm for classifying data from a sample obtained from the individual, wherein the classification includes analyzing the data for the presence, absence or amount of at least measured feature; ii) inputting data from the image sample obtained from the individual; and, iii) classifying the image to indicate the presence, absence, nature or extent of abnormalities.

As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM, and the like; and general hard disks and hybrids of these categories such as magnetic/optical storage media. The medium is adapted or configured for having recorded thereon a marker as described herein.

As used herein, the term “electronic apparatus” is intended to include any suitable computing or processing apparatus or other device configured or adapted for storing data or information. Examples of electronic apparatus suitable for use with embodiments of the present invention include stand-alone computing apparatus; networks, including a local area network (LAN), a wide area network (WAN) Internet, Intranet, and Extranet; electronic appliances such as personal digital assistants (PDAs), cellular phone, pager and the like; and local and distributed processing systems.

As used herein, “recorded” refers to a process for storing or encoding information on the electronic apparatus readable medium. Those skilled in the art can readily adopt any method for recording information on media to generate materials comprising the markers described herein.

A variety of software programs and formats can be used to store the image information on the electronic apparatus readable medium. Any number of data processor structuring formats (e.g., text file or database) may be employed in order to obtain or create a medium having recorded thereon the markers. By providing the markers in readable form, one can routinely access the information for a variety of purposes. For example, one skilled in the art can use the information in readable form to compare a sample image with the control information stored within the data storage means.

Thus, there is also provided herein a medium for holding instructions for performing a method for determining whether a subject has abnormalities, wherein the method comprises the steps of: i) determining the presence or absence of certain features in images, and based on the presence or absence of such features; ii) determining whether the individual has abnormalities, and/or iii) recommending a particular treatment for a particular condition.

It is contemplated that different entities may perform steps of the contemplated methods and that one or more means for electronic communication may be employed to store and transmit the data. It is contemplated that raw data, processed data, diagnosis, and/or prognosis would be communicated between entities which may include one or more of: a primary care physician, patient, specialist, insurance provider, foundation, hospital, database, counselor, therapist, pharmacist, and government.

There is also provided herein an electronic system and/or in a network, a method for determining whether a subject has abnormalities, wherein the method comprises the steps of: i) determining the presence or absence of certain features in images, and based on the presence or absence of such features; ii) determining whether the individual has abnormalities; and/or, iii) recommending a particular treatment for a particular condition. The method may further comprise the step of receiving information associated with the individual and/or acquiring from a network such information associated with the individual.

Also provided herein is a network, a method for determining whether a subject has abnormalities associated with certain features in images, the method comprising the steps of: i) receiving information associated with the images, ii) acquiring information from the network corresponding to images and/or abnormalities, and based on one or more of the images and the acquired information, iii) determining whether the individual has abnormalities. The method may further comprise the step of recommending a particular treatment for the condition.

Systems

Particular embodiments are directed to systems useful for the practice of one or more of the methods described herein. Systems for using detection method described herein for therapeutic, prognostic, or diagnostic applications and such uses are contemplated herein. The systems can include devices for capturing X-ray images, as well as information regarding a standard or normalized profile or control.

Also, the systems can generally comprise, in suitable means for image collecting, devices for each individual image. The kit can also include instructions for employing the kit components as well the use of any other materials not included in the kit. Instructions may include variations that can be implemented. It is contemplated that such reagents are embodiments of systems of the invention. Also, the systems are not limited to the particular items identified above and may include any reagent used for the manipulation or characterization of the images and/or data derived therefrom.

The device and computing system can be configured to process a plurality of images obtained from a single patient imaging session or encounter. Further, it is to be understood that the computing system can be further configured to operate in a telemedicine setting in order to provide clinical health care at a distance. Use of the devices and methods described herein help eliminate distance barriers and can improve access to medical services that would often not be consistently available in distant rural communities, and to receive a request for an analysis from a remote computing system that is in a different geographic location than the computing system.

In certain embodiments, the central processing unit is remotely located from the X-ray machine. In other embodiments, the central processing unit and the X-ray machine can be integrated together in a physical structure (often including a computer and a display screen) that displays information (for example, a physician's office, or in a medical setting such as a clinic, hospital, or the like).

The computing system can be used in the classification of different abnormalities or diseases and to use the set of classifiers to ascertain presence, absence or severity of plurality of diseases, abnormalities, or lesions.

It is further to be understood that the detection of an abnormality can include the gathering of additional data. Non-limiting examples include: demographic information, age data, body mass index data, blood pressure data, genetic information, family history data, race data, systemic factors, and the like.

In a particular embodiment, a computing system can include: one or more hardware computer processors; and one or more storage devices configured to store software instructions configured for execution by the one or more hardware computer processors in order to cause the computing system to: i) determining at least one region-of-interest in an image taken of the subject using an image segmentation procedure; and, ii) classifying the image of step i) using a clique pattern procedure to determine the presence or absence of patterns among at least a first pixel and its adjacent pixels in the region-of-interest in the segmented image; and, based on the presence or absence of such clique patterns, determining whether the subject has such abnormality.

It is also to be understood that the device described herein can be a non-transitory computer storage that stores executable program instructions that, when executed by one or more computing devices, configures the one or more computing devices to perform the operations described herein.

It is also to be understood that the device described herein can be a non-transitory computer-readable medium that stores executable instructions for execution by a computer having memory where the medium storing instructions for carrying out the methods described herein.

In one embodiment, the device can include an X-ray machine constructed to obtain X-ray data, and a central processing unit (CPU) in communication with the X-ray machine. The CPU can include memory-storable CPU-executable instructions for detecting abnormalities.

The CPU can perform the following in response to receiving data based on the memory-storable CPU-executable instructions: a formation of a image based on the X-ray data; an analysis of the image, wherein the analysis comprises: determining the presence or absence of a set of features in at least one image taken from the subject by; i) determining at least one region-of-interest in an image taken of the subject using an image segmentation procedure; and, ii) classifying the image of step i) using a clique pattern procedure to determine the presence or absence of patterns among at least a first pixel and its adjacent pixels in the region-of-interest in the segmented image; and, based on the presence or absence of such clique patterns, determining whether the subject has such abnormality.

The methods and systems of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A computer-implemented method for determining whether a subject has an abnormality present in a cell or tissue, at least a portion of the method being performed by a computing device comprising at least one processor, the method comprises the steps of:
i) determining at least one region-of-interest in an image taken of the subject using an image segmentation procedure; and
ii) classifying the image of step i) using a clique pattern procedure to determine the presence or absence of patterns among at least a first pixel and its adjacent pixels in the region-of-interest in the segmented image;
and, based on the presence or absence of such clique patterns, determining whether the subject has such abnormality.

2. The method of claim 1, wherein the image segmentation procedure comprises using a progressive segmentation of the image to separate the region-of-interest from background in the image.

3. The method of claim 1, wherein the progressive segmentation comprises using:
Fuzzy C-Means Clustering (FCM) which allows data to have different degrees of membership with each clusters; and, White Top-Hat transform which creates different intensity profiles in the image and allows for performing histogram based thresholding.

4. The method of claim 1, wherein the clique pattern procedure comprises using a Gibbs Random Fields (GFRs) clique pattern extraction to search for patterns in the region-of-interest in the image.

5. The method of claim 1, wherein the image includes one or more of: radiographic (X-Ray) images, computer axial tomography (CAT) scans, magnetic resonance images (MRI), and ultrasonic images.

6. The method of claim 1, wherein the abnormality is one or more of: micro-calcifications (MCs), tumors, lesions, injury, tear, or other damage to the tissue or organ.

7. The method of claim 1, wherein the tissues include one or more of blood vessels including small and large arteries, heart valves; joints and tendons including knee joints and rotator cuff tendons; soft tissues including breast, thyroid, testes, muscle, and fat; organs including brain, kidney, bladder, and gallbladder.

8. The method of claim 1, further including indicating when a therapeutic intervention aimed is beneficial.

9. The method of claim 1, further comprising the step of correlating the data with similar data from a reference population.

10. An electronic system for use in determining whether a subject has an abnormality in a cell or tissue, comprising the steps of:
i) determining at least one region-of-interest in an image taken of the subject using an image segmentation procedure; and
ii) classifying the image of step i) using a clique pattern procedure to determine the presence or absence of patterns among at least a first pixel and its adjacent pixels in the region-of-interest in the segmented image;
and, based on the presence or absence of such clique patterns, determining whether the subject has such abnormality.

11. The electronic system of claim 10, further comprising the step of receiving information associated with the subject and/or acquiring from a digital image/acquisition system such information associated with the subject.

12. The electronic system of claim 10, wherein the images are laid down in a database, such as an internet database, a centralized or a decentralized database.

13. The electronic system of claim 10, configured to process a plurality of images obtained from a single patient imaging session or encounter.

14. A computing system comprising:
one or more hardware computer processors; and
one or more storage devices configured to store software instructions configured for execution by the one or more hardware computer processors in order to cause the computing system to perform the method of claim 1.

15. The computing system of claim 14, further comprising a non-transitory computer storage that stores executable program instructions that, when executed by one or more computing devices, configures the one or more computing devices to perform the operations described herein.

16. The computing system of claim 14, further comprising a non-transitory computer-readable medium that stores executable instructions for execution by a computer having memory where the medium storing instructions for carrying out the methods described herein.

17. The computing system of claim 14, further including an image-capturing device constructed to obtain image data, and a central processing unit (CPU) in communication with the image-capturing device.

18. The computing system of claim 14, wherein the CPU includes memory-storable CPU-executable instructions for detecting abnormalities.

19. The computing system of claim 14, wherein the CPU unit is remotely located from the image-capturing device.

20. The computing system of claim 14, wherein the CPU unit and the image-capturing device are integrated together in a physical structure that displays information.

21. A non-transitory computer-readable-storage medium comprising one or more computer-executable instructions, that, when executed by at least one processor of a computing device, causes the computing device to perform the method of claim 1.

22. A network service comprising:
a server connection module configured to receive an image of a subject;
a server processor in data communication with the service connection module for delivery of an evaluation of the image over the network to a network client device; the server processor configured to perform the method of claim 1.

23. The method of claim 1, wherein at least one of steps i) and ii) are performed using a network management server.

24. The method of claim 23, including communicating information about the prediction to a network client device.
